(19) United States
(12) Patent Application Publication
(10) Pub. No.: US 2004/0115666 A1
(43) Pub. Date: Jun. 17, 2004

(54) Method for identifying compounds modulating reverse cholesterol transport

(76) Inventor: Bart Staels, Petit Enghien (BE)

Correspondence Address: NIXON & VANDERHYE, PC, 1100 N GLEBE ROAD, 8TH FLOOR, ARLINGTON, VA, 22201-4714, US

(21) Appl. No.: 10/450,257

(22) PCT Filed: Feb. 4, 2002
(86) PCT No.: PCT/FR02/00410

(30) Foreign Application Priority Data
Feb. 5, 2001 (FR) 01/01486

(51) Int. Cl. C12Q 1/68

(52) U.S. Cl. 435/6

(57) ABSTRACT

The present invention concerns methods and compounds capable of modulating reverse cholesterol transport in a mammal and screening methods for selecting, identifying and/or characterizing compounds capable of modulating reverse cholesterol transport. It also concerns cells, vectors and genetic constructs used for implementing said methods, and pharmaceutical compositions for treating atherosclerosis.

The methods of the invention are based on the use of FXR response elements derived from the apo A-I gene promoter.

The present invention concerns methods and compounds capable of modulating reverse cholesterol transport in a mammal as well as screening methods for selecting, identifying and/or characterizing compounds capable of modulating reverse cholesterol transport. It also concerns cells, vectors and genetic constructs useful for implementing said methods, and pharmaceutical compositions for treating atherosclerosis.

Atherosclerosis is a leading cause of morbidity, mortality, myocardial infarction, cerebral ischemia, cardiovascular disease and peripheral arterial disease. Hypercholesterolemia and cholesterol overload in macrophages, involved in vascular inflammation, are major contributory factors to atherosclerosis. Currently the treatment of hypercholesterolemia consists in a combination of diet and drugs, for example statins or bile acid sequestrants. However, new therapeutic strategies need to be developed to overcome the limitations of existing treatments.

Reverse cholesterol transport, mediated by HDL (high density lipoproteins), removes cholesterol that has accumulated in peripheral tissues and ensures its metabolic clearance in the liver. In this manner it helps protect the body against atherosclerosis. Apolipoprotein A-I (apo A-I) is a basic component of HDL and underlies its efficacy. In this respect, increasing apo A-I expression has a protective effect against atherosclerosis. Apo A-I expression is regulated by hormones or by therapeutic agents such as fibrates. Nuclear receptors such as HNF4, PPARα or RORα have been shown to play a crucial role in the control of apo A-I gene transcription. In particular, PPARα is responsible for the observed increase in apo A-I expression in humans induced by fibrates which find clinical use in the treatment of dyslipidemias. The identification of new intracellular signalling pathways involved in the control of apo A-I expression would therefore make it possible to elaborate new therapeutic strategies capable of increasing the efficiency of reverse cholesterol transport and thus protecting against atherosclerosis.

Nuclear hormone receptors form a large family of transcription factors whose activity can be modulated by natural and/or synthetic ligands. Such transcription factors control the expression of their target genes generally by binding to specific cis-acting response elements and by recruiting accessory proteins required to activate the transcriptional machinery.

The FXR receptor, also known as RIP14 or NR1H4, was originally identified as a famesoid receptor. It acts by forming a heterodimer with the RXR receptor, also a member of this protein family. It was recently shown that several bile acids, such as chenodeoxycholic acid (CDCA) or deoxycholic acid (DCA) and, to a lesser extent, lithocholic acid (LCA), are the true ligands of FXR and that these compounds activate the FXR receptor.

FXR binds preferentially to a response element composed of an inverted repeat of two AGGTCA motifs separated by one nucleotide. It also binds to repeated AGGTCA motifs separated by 2, 4 or 5 nucleotides (DR-2, DR-4, DR-5) (Laffitte B A et al., J. Biol. Chem., 2000, 275: 10638-10647). Several target genes of FXR have been identified, such as Cyp7α-hydroxylase (see WO 00/40965), I-BABP, PLTP and CPT-II.

The present invention is based on the observed role of FXR in the expression of the human apo A-I gene and on the direct interaction which occurs between FXR and two distinct fragments of the promoter of this gene. It is also based on the original observation that overexpression of FXR in the presence of bile acids causes repression of human apo A-I promoter activity. It is further based on the identification of sites C and A in the human apo A-I gene promoter as being functional FXR response elements and the characterization of their respective sequences. It is also based on the original observation that FXR bound to site C inhibits apo A-I expression, that FXR also binds to this site in an unexpected and unforeseeable manner as a monomer and that inhibition of the activity of the human apo A-I gene promoter by FXR via site C predominates over its activating action via site A.

The present invention thus provides the first demonstration of modulation of apo A-I production by the FXR nuclear receptor. The present invention thus discloses new targets and new approaches by which to search for compounds capable of regulating the expression of this protein, or the activity of HDL, or reverse cholesterol transport.

The invention also describes methods for increasing reverse cholesterol transport based on using compounds that modulate FXR binding to the apo A-I promoter and/or the effect thereof on the transcription of the human apo A-I gene.

The invention further provides screening methods to identify therapeutic substances capable of modulating the expression of the human apo A-I gene and/or the activity of HDL and/or reverse cholesterol transport.

According to a particular embodiment, the screening methods of the invention more specifically include the following steps:

contacting one or more compounds with a nucleic acid construct containing at least one FXR response element of the human apo A-I gene promoter or a functional variant thereof, in conditions suitable for allowing said compounds to bind to said response element,

determining the possible binding of said compounds to the response element(s), and

optionally comparing the previous measurement with a measurement carried out in the same conditions but with a nucleic acid construct containing at least one mutated copy of an FXR response element of the human apo A-I gene promoter.

According to a particular embodiment of the method of the invention, the conditions suitable for allowing said compounds to bind to said FXR response element(s) comprise the presence of the FXR receptor (for example, in monomeric form) or of a functional equivalent, and determining the effect of the presence of the test compound on FXR binding to the response element.

According to another specific embodiment (test of transcriptional activity), one measures the effect of one or more test compounds on the transcriptional activity of a promoter containing at least one FXR response element according to the invention. Such test is preferably carried out in a cellular system, by measuring the expression of a reporter gene placed under the control of such promoter, particularly in a cell comprising (e.g., expressing, naturally or through recombination) the FXR receptor or a functional equivalent thereof.

A preferred embodiment of the invention consists in using an expression cassette combining one or more FXR response elements, according to the invention, with a reporter gene. In an advantageous manner, said reporter gene is placed under the control of a promoter containing at least one copy of said response element(s), for example, the apo A-I promoter or variants or fragments thereof. Any gene known to those skilled in the art whose activity or presence in biological extracts is easily measurable can be used as reporter gene to carry out the screening method.

The compounds that can be identified by the method of the invention may be compounds of different nature, structure and origin, particularly biological compounds, nuclear factors, cofactors, and the like, chemical, synthetic compounds and the like, capable of modifying the activity of FXR. They may also be libraries, particularly libraries of chemical compounds or libraries of proteins, peptides or nucleic acids, for instance clones encoding DNA-binding proteins or peptides.

The methods according to the invention may be used to select, identify or characterize compounds capable of modifying the binding of FXR to one and/or the other of its response element(s) and/or modulating (that is, increasing or decreasing) the expression of the human apo A-I gene and/or modulating the activity of HDL and/or modulating reverse cholesterol transport.

The invention further describes the use of compounds thereby selected, for preparing a composition for modulating reverse cholesterol transport or HDL activity, and the corresponding methods of treatment.

For easier comprehension of this application, the following definitions are given, which clarify or add to their usual meaning.

“Apolipoprotein A-I” or apo A-I: Apolipoprotein A-I is a protein of 243 amino acids with an amino terminal globular domain and a carboxy terminal capable of binding to lipids (Segrest et al., Curr. Opin. Lipidol., 2000, 11: 105-115). This protein is a major constituent of high density lipoproteins and plays a fundamental role in reverse cholesterol transport (Fruchart and Duriez, Biochimie, 1998, 80: 167-172, Leroy et al., Curr. Opin. Lipidol., 1995, 6: 281-285). The apo A-I gene, cDNA and mRNA have been cloned and sequenced (Breslow et al., PNAS, 1982, 79: 6861-6865, Shoulders and Baralle, Nuc. Ac. Res., 1982, 10: 4873-4882, Karathanasis et al., Nature, 1983, 304: 371-373), and are accessible in the Genbank* database (for example at the following internet address: http://www.ncbi.nlm.nih.gov) under access numbers: NM_{—}000039, M20656 (promoter) and J00098).

“High Density Lipoprotein (HDL)”: HDL particles are high density lipoproteins (1.063-1.21 g/ml) known to play a protective role against atherosclerosis, primarily due to their ability to extract cholesterol from peripheral cells and promote its transport to the liver where it is eliminated (Fruchart and Duriez, Biochimie, 1998, 80: 167-172). Apo A-I is the major protein component of HDL, representing up to 70% of the protein. HDL also includes apo A-II, apo C-I, apo C-II, apo C-III and apo E in lower proportions.

“FXR”: The FXR receptor has been isolated, characterized and sequenced in man and mouse (Forman et al., Cell, 1995, 81: 687-693, Seol et al., Mol. Endo., 1995, 9: 72-85). The mRNA sequence is also available in the Genbank® database (access numbers NM_{—}005123, NM_{—}021745 and U18374). The FXR DNA binding domain is comprised mainly between residues Cys 124-Met 189 of the human protein (corresponding to 726-953 in NM_{—}005123) or between residues 423-621 (U1 8374) of the murine protein.

The expression “functional equivalent” which refers to the FXR receptor denotes any polypeptide derived from the structure of the FXR receptor and conserving the capacity to bind to the response element, particularly any response element having sequences SEQ ID NO: 1 or 2 or functional variants thereof. Functional equivalents may be natural variants (polymorphism, splicing, etc.), fragments, mutants, deletions, etc. They are preferably polypeptides containing at least one region of amino acids sharing at least 60% identity to those of the FXR receptor, preferably at least 75% and even more preferably at least 90-95%. The expression also includes fragments of the FXR receptor, particularly fragments containing the DNA binding site of the FXR receptor.

The term “reverse transport” is used to denote the physiological mechanism, sometimes defective, whereby excess cholesterol in peripheral tissues is collected by high density lipoprotein (HDL) and then transported to the liver where it is eliminated.

A. Identification of an FXR Response Element.

The present invention demonstrates the involvement and the mechanism of action of FXR in the regulation of apo A-I expression by bile acids and, this being the case, in the regulation of reverse cholesterol transport. In the presence of bile acids, overexpression of FXR results in a reduction of the activity of the latter. The invention further discloses the exact sequence of two FXR response elements located in the promoter of the human apo A-I gene.

The invention also relates to specific constructs, particularly nucleic acids comprising FXR response elements, as well as cassettes, vectors and recombinant cells containing them.

Thus the invention discloses the sequences (SEQ ID NO: 1 and SEQ ID NO: 2) of two FXR response elements, initially identified in the human apo A-I gene promoter, responsible for an interaction between FXR and the apo A-I promoter and for FXR regulation of apo A-I expression.

For example, the presence of three copies of site C (SEQ ID NO: 1) causes inhibition by FXR and bile acids of the expression of a reporter gene (see example 5). Conversely, the presence of three copies of site A (SEQ ID NO: 2) causes stimulation of said expression (see example 5).

A particular object of the invention is a nucleic acid comprising sequence SEQ ID NO: 1 or 2, or a functional variant thereof (“FXR response element”).

Another object of the invention is a nucleic acid construct comprising an FXR response element as defined hereinabove. In particular this may be an expression cassette containing at least one copy of a response element such as defined hereinabove.

The invention further concerns any artificial or chimeric promoter containing an FXR response element such as defined hereinabove.

The functional variants of the response element according to the invention may be any derivative or fragment of the native sequence conserving the capacity to bind to the FXR receptor. In general, the variants conserve at least 50% of the residues of the native sequence described in the present application. Classically, the variants contain modifications affecting fewer than 5 nucleotides in the sequence under consideration. They preferably show at least 60% sequence identity, preferably at least 75% and even more preferably at least 90% sequence identity to the native sequence described in the present application.

The variants may contain different types of modifications such as one or more point mutations or not, additions, deletions and/or substitutions.

Such modifications may be introduced by conventional physical, chemical or molecular biological methods, such as in particular site directed mutagenesis or, more practically, by artifical synthesis of the sequence in a synthesizer.

The variants may be tested for their capacity to bind FXR in different ways, and particularly:

(i) by placing the test sequence in contact with the FXR receptor (for example in an acellular test), and detecting the formation of a complex (for example by gel shift assay);

(ii) by inserting the test sequence in an expression cassette containing a minimal promoter and a reporter gene, introducing the cassette into a cell, and detecting (assaying, as the case may be) the expression of the reporter gene in the presence and absence of FXR;

(iii) by any other method known to those skilled in the art, allowing to demonstrate an interaction between a nucleic acid and a protein, for example.

A further object of the invention consists of inactive variants of the hereinabove defined response elements, particularly variants essentially incapable of binding the FXR receptor. Such variants are exemplified in particular by sequences SEQ ID NO: 3 and 4.

Such inactive variants may be prepared and tested under the conditions described hereinabove for functional variants.

The variants according to the invention advantageously are capable of hybridizing with sequence SEQ ID NO: 1 or 2 or a part thereof.

B. Methods for Selecting, Identifying and Characterizing Compounds Modulating Reverse Cholesterol Transport.

The invention describes methods for identifying compounds which modulate (that is, increase or decrease) reverse cholesterol transport. Such compounds may act by altering FXR binding to its ligand or ligands (bile acids, co-repressors and co-activators, etc.). They may futher modify, even suppress, the binding of FXR alone or of FXR and its cofactors, to its response element(s) and thereby modify the expression of the human apo A-I gene. For instance, binding of FXR to the response element C (SEQ ID NO: 1) decreases transcription of the human apo A-I gene and reduces reverse cholesterol transport. The use of compounds that can inhibit the binding of FXR to this response element, where FXR plays the role of repressor, therefore in contrast allows to increase transcription of the human apo A-I gene and to stimulate reverse cholesterol transport.

The present invention also demonstrates in a surprising manner that binding of FXR to response element A (SEQ ID NO: 2) increases the transcription of the human apo A-I gene and increases reverse cholesterol transport. The use of compounds capable of stimulating FXR binding to this response element, where in this case FXR acts as a transcriptional activator (or of reproducing this activation by direct binding), therefore also allows to increase transcription of the human apo A-I gene and to stimulate reverse cholesterol transport.

The present invention thus describes new methods for selecting, identifying or characterizing compounds capable of increasing reverse cholesterol transport.

1. Methods Based on Screening of Expression.

The present invention concerns a method for selecting, identifying or characterizing compounds capable of modulating reverse cholesterol transport, which comprises:

contacting a test compound with a host cell containing an expression cassette of a reporter gene, said cassette comprising a reporter gene placed under the control of a promoter containing at least one copy of an FXR response element of the human apo A-I gene promoter or a functional variant thereof, and

measuring the expression of the reporter gene.

The methods according to the invention more specifically provide for contacting a test compound with a nucleic acid construct or an expression cassette containing at least one copy of an FXR response element (SEQ ID NO: 1 or 2). According to another embodiment of the invention, the copy of the FXR response element(s) (site C and/or A) may be a mutated copy, said mutated copy being essentially incapable of binding the FXR receptor, even in the presence of bile acids.

According to a particular embodiment of the methods of the invention, it is further provided to compare the possible effects determined by means of one of these methods, with the possible effects determined by means of a method carried out in the same conditions but with a nucleic acid construct containing at least one inactive variant (for instance, a mutated copy) of an FXR response element of the human apo A-I gene promoter (SEQ ID NO: 1 or 2) or a functional variant of the latter.

The methods of the invention may be carried out with different types of cells, promoters, reporter genes, and in different conditions, as described hereinafter.

a) Contacting the Compounds With the Host Cell

Some screening methods, described by the invention, thereby provide for a step of contacting the test compound with host cells, in specific conditions allowing to measure the expression in said cells of a reporter gene and thus to obtain information concerning the effect of the test compound. Classically, the effect of the test compound is compared to the level of expression of a reporter gene measured in the absence of said compound (and/or with a mutated response element).

In a preferred embodiment of the invention, such cells may be mammalian cells (hepatocytes, fibroblasts, endothelial cells, muscle cells, etc.). Even more preferably, such cells may be human cells. They may also be primary cultures or established cell lines. In another embodiment, it is also possible to use prokaryotic cells (bacteria), yeast cells (Saccharomyces, Kluyveromyces, etc.), plant cells, and the like.

The compounds may be placed in contact with the cells at different times, according to their effect(s), their concentration, the type of cells and technical considerations. The contact may be carried out on any suitable support and particularly on a plate, in a tube or a flask. Generally, placing in contact is carried out in a multiwell plate which makes it possible to concurrently conduct many different tests. Typical supports include microtitration plates and more particularly plates containing 96 or 384 wells (or more), easy to manipulate and on which visualization may be accomplished through a classical stimulation.

Depending on the support and the nature of the test compound, variable quantities of cells may be used to carry out the methods described herein. Classically, 10³ to 10⁶ cells are contacted with a type of test compound, in a suitable culture medium, and preferentially between 10⁴ and 10⁵ cells. By way of example, in a 96 well plate, 10⁵ cells can be incubated in each well with a desired quantity of test compound. In a 384 well plate, less than 10⁵ cells and typically between 1×10⁴ and 4×10⁴ cells are generally incubated in each well with the test compound.

The quantity (or concentration) of test compound may be adjusted by the user according to the type of compound (its toxicity, its ability to penetrate into the cell, etc.), the number of cells, the incubation time, etc. Generally, cells are exposed to concentrations of test compounds which range from 1 nM to 1 mM. Of course other concentrations may be tested without deviating from the present invention. Each compound may furthermore be tested at different concentrations concurrently. Different adjuvants and/or vectors and/or products that facilitate penetration of compounds inside cells such as liposomes, cationic lipids, polymers, penetratin, Tat PDT, peptides from adenovirus (penton or fibers) or other viruses, and the like, may additionally be used if necessary.

Contact is maintained for between 5 to 72 hours, generally between 12 and 48 hours. In fact, the cells and the various reagents should preferably remain in contact long enough to allow de novo synthesis of the expression product of the reporter gene. In a preferred manner, the incubation time is approximately 36 hours.

b) Determining the Activity of the Compounds.

The method provided for in the invention to select, identify or characterize compounds capable of modulating reverse cholesterol transport calls for the transformation of the host cells with a reporter gene expression cassette. Said reporter gene may in particular be any gene whose transcription or expression product can be detected or assayed in biological extracts. For example, it may be the gene coding for human apo A-I itself, or yet the gene coding for luciferase and more particularly for firefly or Renilla luciferase, for secreted alkaline phosphatase, galactosidase, lactamase, chloramphenicol acetyl transferase (CAT), human growth hormone (hGH), β-glucuronidase (Gluc) and green fluorescent protein (GFP), etc. It is understood that the term “gene” denotes in the broad sense any nucleic acid, particularly a cDNA, a gDNA, a synthetic DNA, an RNA, etc.

The reporter gene, whatever it may be, is placed under the control of a promoter containing at least one copy of an FXR response element such as described hereinabove.

The reporter gene may therefore be placed under the control of any promoter whose sequence comprises one of the sequences SEQ ID NO: 1 and/or 2 or a functional variant thereof. Such specific sequence may be present in one or more copies in the promoter (preferably 1 to 10 and even more preferably 1 to 6), upstream, downstream, or inside, in the same orientation or in the opposite orientation. In a preferred embodiment of the invention, the reporter gene is placed under the control of a promoter which contains one or more copies of site A (SEQ ID NO: 2) and no site C. In another embodiment of the invention, the reporter gene is placed under the control of a promoter which contains one or more copies of site C (SEQ ID NO:1) and no site A. In a further embodiment of the invention, the reporter gene is placed under the control of a promoter which contains one or more copies of sites C and A. Preferably, the promoter is one whose differential activity in the absence and presence of FXR or of a functional equivalent can be detected.

To construct a promoter of the invention, the FXR response element may be associated with a transcriptional minimal promoter. The minimal promoter is a transcriptional promoter having a low or absent basal activity, and able to be increased in the presence of a transcriptional activator (interaction of FXR in the presence of bile acids with site A). A minimal promoter may therefore be a naturally weak promoter in mammalian cells, that is, producing a nontoxic and/or insufficient expression to obtain a marked biological effect. Advantageously, a minimal promoter is a construct prepared from a native promoter by deletion of region(s) which are not essential for transcriptional activity. Thus, it is preferably a promoter containing essentially a TATA box, generally less than 160 nucleotides in length, centered around a transcription initiation codon. A minimal promoter can thus be prepared from strong or weak viral or cellular promoters, examples of which include the promoter of the herpes virus thymidine kinase (TK) gene, the CMV immediate promoter, the PGK promoter, the SV40 promoter, etc. The minimal promoter may have sufficiently high activity to allow identification of compounds which increase FXR-mediated inhibition, via site C, or increase FXR-mediated activation, via site A, for instance.

The promoter (P), the FXR response element (RE) and the reporter gene (RG) are arranged in a functional manner in the expression cassette, that is, in such a way that the minimal promoter controls the expression of said gene and that its activity is regulated by FXR. Generally, such regions are therefore arranged in the following order, in the 5′→3′ direction: RE-P-RG. However, any other functional arrangement may be used by those skilled in the art without deviating from the present invention.

Moreover, the different functional domains hereinabove may directly flank each other or be separated by nucleotides having no significant effect on the functional character of the expression cassette or allowing to confer improved characteristics or performance of the system (amplifier, silencer, intron, splicing site, etc.).

The method for selecting, identifying and characterizing compounds capable of modulating reverse cholesterol transport provides for a step to measure the expression of the reporter gene. This may be a measurement of transcriptional activity. To this end, total RNA is extracted from the cells in culture in experimental conditions on the one hand and in a control situation on the other hand. Such RNA is used as probe to analyze, for example, the changes in the expression of the reporter gene(s).

This may also involve a visualization of expression of the reporter gene by means of a suitable substrate. Such visualization may be accomplished by different methods whose nature depends on the type of reporter gene used. For instance, the measurement may correspond to an optical density or fluorescence emission in the case of using the β-galactosidase or luciferase gene as reporter gene.

In a specific embodiment, expression of the reporter gene is measured by hydrolysis of a substrate of the expression product of the reporter gene. For instance, a number of substrates may be used to evaluate the expression of β-lactamase. In particular these may be any products containing a β-lactam nucleus and whose hydrolysis can be controlled. Preferred substrates are those specific for β-lactamase (i.e., generally they are not hydrolyzed by mammalian cells in the absence of β-lactamase), those which are not toxic to mammalian cells and/or whose hydrolysis product may be easily measured, for example by methods based on fluorescence, radioactivity, enzymatic activity or any other means of detection. Even more preferred substrates are radiometric substrates. Hydrolysis of such substrates may be directly related to the activity of the expression product of the reporter gene by the number of cells. A specific and nontoxic radiometric substrate usable in the present invention is CCF2-AM.

The concentration of the substrate may be adjusted by those skilled in the art according to the number of cells, for example. The cells are generally contacted with the substrate for approximately 60 minutes.

The presence of the reporter gene product (or hydrolysis product of the substrate) may be determined by conventional methods known to those skilled in the art (fluorescence, radioactivity, optical density, luminescence, FRET (see WO 0037077), SPA, biochips, immunological methods, etc.).

Generally, one determines the activity of a test compound in a cell and compares this effect to the level of activity in the absence of test compound or to a mean value determined in the absence of any test compound.

Measuring hydrolysis essentially means measuring (or determining the relative amount of) the hydrolysis product contained in each reaction sample. Such measurement may be carried out by different methods known to those skilled in the art, including detection of fluorescence, radioactivity, color, enzymatic activity, an antigen-antibody immune complex, and the like. In a preferred manner, the hydrolysis product is detected and quantified by a fluorescence detection method. Different fluorochromes may thus be used and controlled on the cell samples.

A secondary test allowing to validate the selected compounds in animals may also be performed by determining the amount of HDL expressed or by determining a significant variation in reverse cholesterol transport in cells treated with said compounds as compared to untreated cells. It is also possible to measure plasma cholesterol and/or determine hepatic expression of apo A-I.

In a preferred manner, the method according to the invention makes use of a host cell containing the FXR receptor or a functional equivalent thereof.

The presence of the FXR receptor allows to reproduce the physiological situation and to identify, through the hereinabove described methods, compounds capable of modulating interactions between FXR and one and/or the other of its response element(s), as disclosed by the present invention, or between FXR and one or more FXR ligand(s).

The FXR receptor may be naturally present or may have been introduced or added by artificial means. It may be an FXR equivalent, meaning that it shares at least 60% amino acid identity to the FXR receptor, preferably at least 75% and even more preferably at least 90-95%.

According to a preferred embodiment, the invention also describes a method for selecting, identifying or characterizing compounds capable of modulating reverse cholesterol transport, which comprises:

contacting a test compound with a host cell containing:

an expression cassette of a reporter gene, said cassette comprising a reporter gene placed under the control of a promoter containing at least one copy of an FXR response element of the human apo A-I gene promoter or a functional variant thereof, and

the FXR receptor or a functional equivalent thereof, and

measuring the expression of the reporter gene.

This method makes it possible to determine the level of expression of the reporter gene according to one of the hereinabove described methods known to those skilled in the art, in the presence of the test compound or in the absence of said compound, an increase or decrease in the level of reporter gene expression being a reflection of the ability of the test compound to modulate reverse cholesterol transport.

According to an even more preferred embodiment of the invention, the host cell also contains an FXR ligand.

The term “FXR ligand” applies not only to bile acids and derivatives but also to transcription factors, co-activators and co-repressors, as well as to other polypeptides involved in the regulatory machinery of gene expression. For example, these may be other receptors such as RXR or nuclear hormone receptors.

As indicated hereinabove, such methods enable rapid and simultaneous screening of many test compounds on one or more cell populations (mammalian cells, human cells such as hepatocytes for example, prokaryotic cells, etc.). These methods are predictive and can be automated and are suitable for selecting, identifying and characterizing said compounds.

A particular embodiment of the screening method makes use of conventional methods for identifying clones which express DNA-binding proteins. This may involve for instance screening γgt11 cDNA expression libraries or using the so-called “One Hybrid” or “Phage Display” method, or yet carrying out a purification by affinity chromatography. The isolated protein or proteins are then sequenced.

2. Methods Based on a Binding Test.

The invention also concerns a method for selecting, identifying or characterizing compounds capable of modulating reverse cholesterol transport, based on measuring the binding of a test compound to one or the other or even both response elements. Such method more particularly comprises:

contacting a test compound with a nucleic acid construct containing at least one copy of an FXR response element of the human apo A-I gene promoter or a functional variant thereof, and

determining the possible binding of said test compound to said response element.

Binding of the test compound to at least one FXR response element may be demonstrated by electrophoretic gel migration of heterodimers formed as a result of implementation of the method described hereinabove. As a matter of fact, some test compounds may harbor a DNA binding site largely identical to that of FXR and so may act as competitors of the latter.

Electrophoresis makes it possible to directly distinguish heterodimers of FXR /FXR response element, from heterodimers of test compound/FXR response element and from FXR response elements.

Other methods based on luminescence or employing the FRET (Fluorescence Resonance Energy Transfer) method, well known to those skilled in the art, or the SPA (Scintillation Proximity Assay) method, may be utilized within the scope of the present invention to determine the potential binding of the test compound to one and/or the other FXR response element(s).

This direct method for selecting, identifying and characterizing compounds capable of modulating reverse cholesterol transport may, in a particular embodiment of the present invention, be carried out in the presence of the FXR receptor or a functional equivalent of the latter. The final step of such method then consists in determining the effect of the presence of the test compound on binding of FXR to its response element(s). For example, this establishes the capacity of said test compound to modulate FXR binding to the response element, by measuring the quantity of FXR bound in the presence of the test compound relative to the quantity in the absence of the test compound. A competition test utilizing the fluorescence polarization (FP) method known to those skilled in the art may thus be efficiently used for the purposes of such measurement.

A test compound capable of modulating FXR binding to the response element may be the object of a subsequent test of its capacity to modulate expression of a reporter gene and/or reverse cholesterol transport, according to one of the methods described hereinabove.

In a specific embodiment, the nucleic acid construct contains at least 1 copy, preferably 2 to 5 copies of the sequence SEQ ID NO: 1 or a functional variant thereof. Test compounds capable of inhibiting (that is, reducing at least partially) the binding of FXR to this construct allow to activate the expression of the reporter gene and constitute candidates for stimulation of reverse cholesterol transport.

C. Activity of HDL and Apolipoprotein A-I.

The methods described hereinabove for selecting, identifying and characterizing compounds capable of modulating the expression of a reporter gene and/or reverse cholesterol transport may, in another embodiment of the invention, be used to select, identify and characterize compounds capable of modulating the activity of HDL and/or the expression of apo A-I.

D. Test Compounds.

The present invention may be put to use with any type of test compound. For example, the test compound may be any product used alone or in a mixture with other products. The compound may be defined in terms of its structure and/or composition or may not be defined. For instance, the compound may be an isolated and structurally elucidated product, an isolated product with undefined structure, a mixture of known, characterized products, or an undefined composition comprising one or more products. Such undefined compositions may be exemplified by tissue samples, biological fluids, cellular supernatants, plant preparations, and the like. The test compounds may be inorganic or organic products and particularly a polypeptide (or a protein or a peptide), a nucleic acid, a lipid, a polysaccharide, a chemical or biological compound such as a nuclear factor, a cofactor or any mixture or derivative of the latter. The compound may be natural or synthetic and include a combinatorial library, a clone or a library of nucleic acid clones expressing one or more DNA-binding peptide(s), etc.

The present invention is particularly suitable for selecting, identifying or characterizing a large number of compounds. Such simple and efficient screening may be accomplished in a very short time. In particular, the methods described herein may be partially automated, thereby making it possible to efficiently and simultaneously screen many different compounds, either as a mixture or individually.

E. Use of the Identified Compounds.

The compounds identified according to the invention display properties that are advantageous with a view to therapeutic use, particularly in the field of atherosclerosis. One possible application of the invention is to enable identification of a therapeutic substance capable of reducing the inhibitory effect of bile acids on apo A-I expression and thus increasing reverse cholesterol transport. The invention thus provides for the use of a compound capable of modulating (that is, increasing or decreasing) FXR binding to response elements of the human apo A-I gene promoter or a functional variant thereof, for preparing a composition for modulating (that is, increasing or decreasing) reverse cholesterol transport. According to another embodiment of the invention, such utilization may be for modulating (that is, increasing or decreasing) the activity or HDL or for modulating the expression of apo A-I.

A further embodiment of the invention provides for the use of a compound capable of controlling the effect of FXR on transcription of the human apo A-I gene or a functional variant therof, for preparing a composition for increasing reverse cholesterol transport.

According to a preferred embodiment of the invention, the compound is a chemical compound or a biological compound. In another preferred embodiment, the compound is a nuclear factor or a cofactor. In an even more preferred embodiment, it is a clone expressing one or more DNA-binding polypeptide(s). In general, it may be any compound selected, identified or characterized according to one of the hereinabove described methods.

The invention encompasses the use of any compound (or derivatives of said compounds) selected, identified or characterized according to one of the methods hereinabove described, within the scope of the present invention, as target of experimental research or for preparing pharmaceutical compositions for increasing reverse cholesterol transport or treating hypercholesterolemia, atherosclerosis, lipid disorders and/or cardiovascular diseases.

Other advantages and applications of the present invention will become apparent in the following examples, which are given for purposes of illustration and not by way of limitation.

### LEGENDS OF FIGURES

FIG. 1: TCA reduces plasma concentrations of cholesterol and apo A-I as well as hepatic expression of apo A-I in mice transgenic for the human apo A-I gene.

FIG. 2: CDCA and TCA reduce apo A-I expression in HepG2 cells.

FIG. 3: Transcriptional effect of bile acids.

FIG. 4: CDCA and TCA activate FXR.

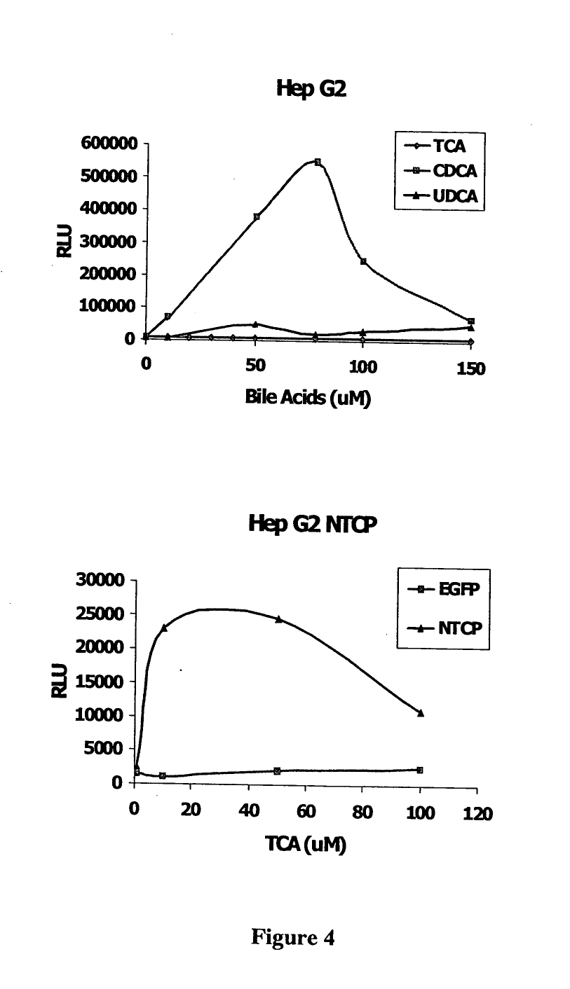

FIG. 5: Overexpression of FXR in the presence of CDCA reduces human apo A-I promoter activity in HepG2 cells.

FIG. 6: Overexpression of FXR in the presence of CDCA reduces human apo A-I promoter activity in HepG2 cells: functional identification of response elements.

FIG. 7 : FXR binds to sites C (SEQ ID NO: 1) and A (SEQ ID NO: 2) of the apo A-I promoter as a dimer with RXR whereas FXR also binds as a monomer to site C (SEQ ID NO: 1).

FIG. 8: GW4064, a specific activator of FXR, redues human apo A-I expression as well as its promoter activity in HepG2 cells.

### SEQUENCES

| | |
|---|---|
| (Site C wt) | |
| 5′-CAGAGCTGATCCTTGAACTCTTAAGTTCC-3′ | SEQ ID NO:1 |
| (Site A wt) | |
| 5′-CCCACTGAACCCTTGACCCCTGCCCTGCAGCC-3′ | SEQ ID NO:2 |
| (Site C mt) | |
| 5′-CAGAGCTATATATTATATATATAAGTTCC-3′ | SEQ ID NO:3 |
| (Site A mt) | |
| 5′-CCCCACTGAACCCTTGATTCCTGCTTTGCAGCC-3′ | SEQ ID NO:4 |
| (apo A-I promoter-j04066 (apo A-I gene) 1819-2167) | |
| 5′-gggagacctgcaagcctgcagcactcccctcccgcccccactgaacccttgacccctgccctgcagcccccgca | SEQ ID NO:5 |
| gcttgctgtttgcccactctatttgcccagccccagggacagagctgatccttgaactcttaagttccacattgccagg | |
| accagtgagcagcaacagggccggggctgggcttatcagcctcccagcccagaccctggctgcagacataaata | |
| ggccctgcaagagctggctgcttagagactgcgagaaggaggtgcgtcctgctgcctgccccggtcactctggct | |
| ccccagctcaaggttcaggccttgccccaggccgggcctctgggtac-3′ | |
| (TK promoter-M80483 (pBLCAT5) 38-204; J02224 (Herpes | |
| simplex) 302-462) | |
| 5′-tgccccgcccagcgtcttgtcattggcgaattcgaacacgcagatgcagtcggggcggcgcggtccaggtccact | SEQ ID NO:6 |
| tcgcatattaaggtgacgcgtgtggcctcgaacaccgagcgaccctgcagcgacccgcttaacagcgtcaacacg | |
| tgccgcagatcacgag-3′ | |

### EXAMPLES

### Example 1

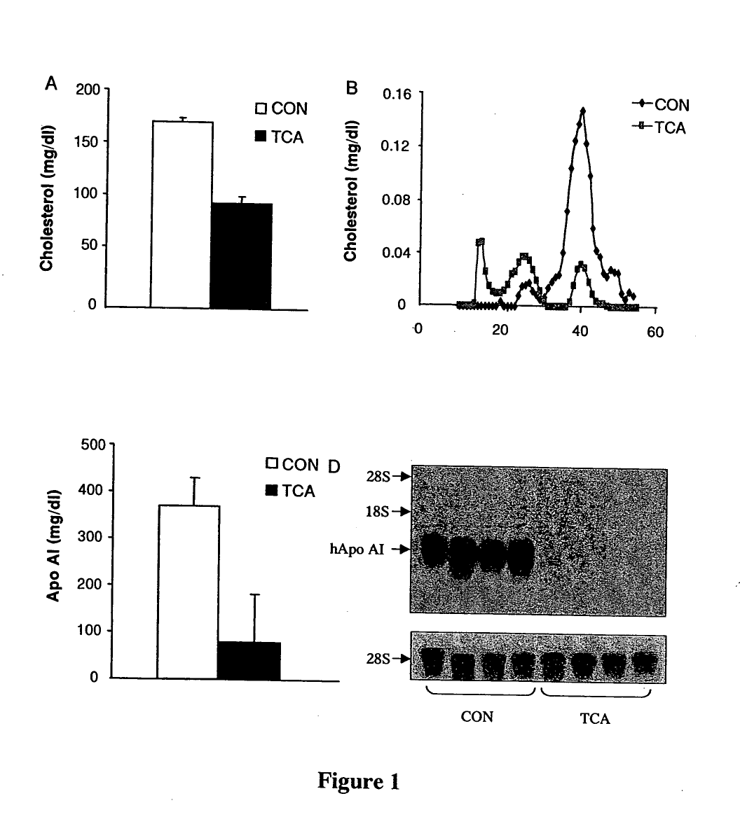

Effect of TCA on Plasma Concentrations of Cholesterol and Apo A-I and on Hepatic Expression of apo A-I in Mice Transgenic for the Human apo A-I Gene (FIG. 1)

Example 1 illustrates the effect of a diet rich in taurocholate on serum cholesterol and human apo A-I levels and on lipid profile and hepatic expression of human apo A-I in transgenic mice expressing the apo A-I gene in a C57BL genetic background.

Ten transgenic mice expressing the apo A-I gene in a C57BL genetic background (IFFA-CREDO, L'Arbresle, France) were genotyped by PCR and divided into two groups of five animals. The first group received a normal diet for one week while the second was given a diet rich in TCA (taurocholic acid, 0.5% m/m). Blood was collected after a 12 hour fast by retroorbital puncture under barbital anesthesia. Serum samples were collected and stored at −20° C. pending analysis. After one week of dietary treatment, animals were sacrificed and liver specimens were collected and frozen for analysis. Human apo A-I levels in serum were measured before and after the diet as previously described (Peters, J. Biol. Chem., 1997, 272 (43): 27307-12). Serum cholesterol and lipid profiles after FPLC were measured as previously described (Raspé et al., J. Lipid Res., 1999, 40 (11): 2099-2110). Total RNA was extracted from liver specimens as previously described (Chomczynski et al., Anal. Biochem., 1987, 162 (1): 156-9) and quantified by Northern Blot hybridization (Peters, 1997) using human apo A-I and 28S as probes (Peters, J. Biol. Chem., 1997, 272 (43): 27307-12).

### Example 2

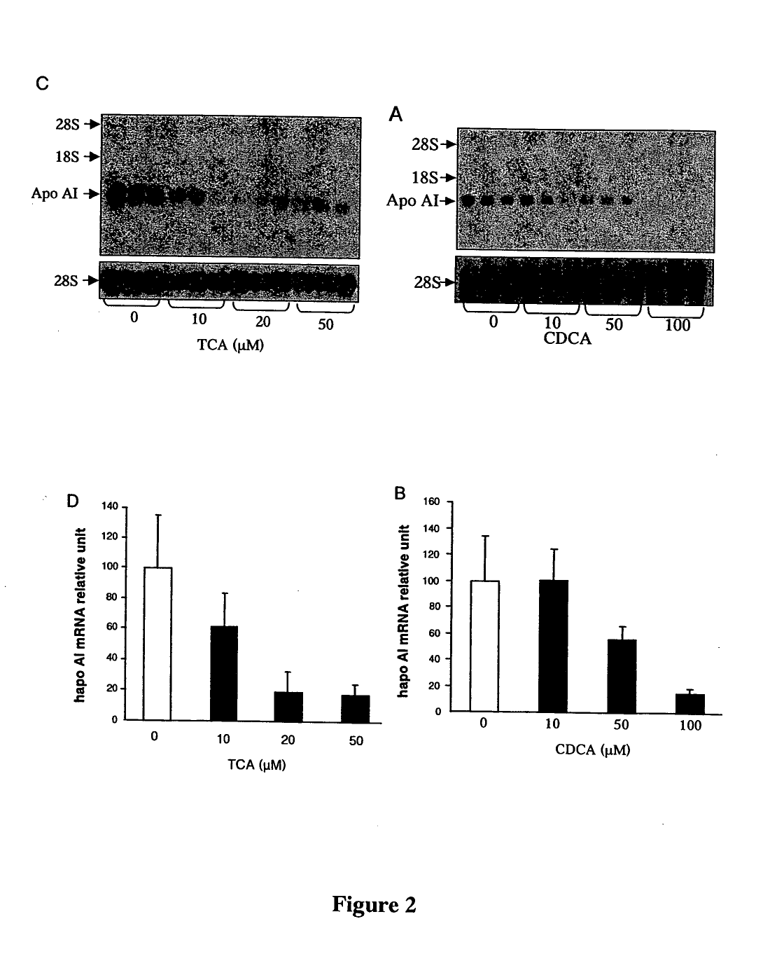

Effect of CDCA and TCA on Apo A-I Expression in HepG2 Cells (FIG. 2)

Example 2 shows the effects of increasing concentrations of CDCA and TCA which reduce apo A-I expression in HepG2 cells. HepG2 cells were grown in DMEM medium supplemented with 10% fetal calf serum, penicillin/streptomycin, sodium pyruvate and nonessential amino acids and incubated at 37° C in a humidified 5% CO₂/95% air atmosphere. They were then treated with the indicated concentrations of CDCA. Total RNA was extracted as previously described (Chomczynski et al., Anal. Biochem., 1987, 162 (1): 156-9) and quantified by Northern Blot hybridization (Peters, J. Biol. Chem., 1997, 272 (43): 27307-12) using human apo A-I and 28S as probes (Peters, J. Biol. Chem., 1997, 272 (43): 27307-12).

### Example 3

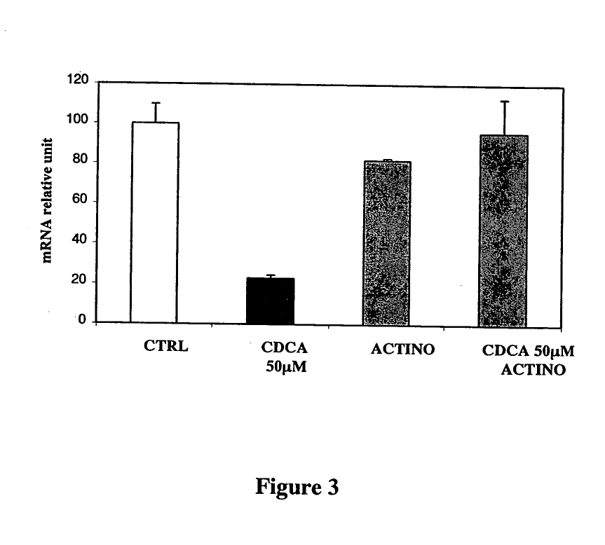

Demonstration of a Transcriptional Effect of Bile Acids (FIG. 3)

Example 3 shows that CDCA at a concentration of 50 μM considerably reduces apo A-I expression in HepG2 cells. Its effect is offset by a 12-h preincubation with actinomycin D (5 g/ml), which suggests that the effect occurs at the level of transcription. HepG2 cells were grown in DMEM medium supplemented with 10% fetal calf serum, penicillin/streptomycin, sodium pyruvate and nonessential amino acids and incubated at 37° C. in a humidified 5% CO₂/95% air atmosphere. Total RNA was extracted as previously described (Chomczynski et al., Anal. Biochem., 1987, 162 (1): 156-9) and quantified by Northern Blot hybridization (Peters, J. Biol. Chem., 1997, 272 (43): 27307-12) using human apo A-I and 28S as probes (Peters, J. Biol. Chem., 1997, 272 (43): 27307-12).

### Example 4

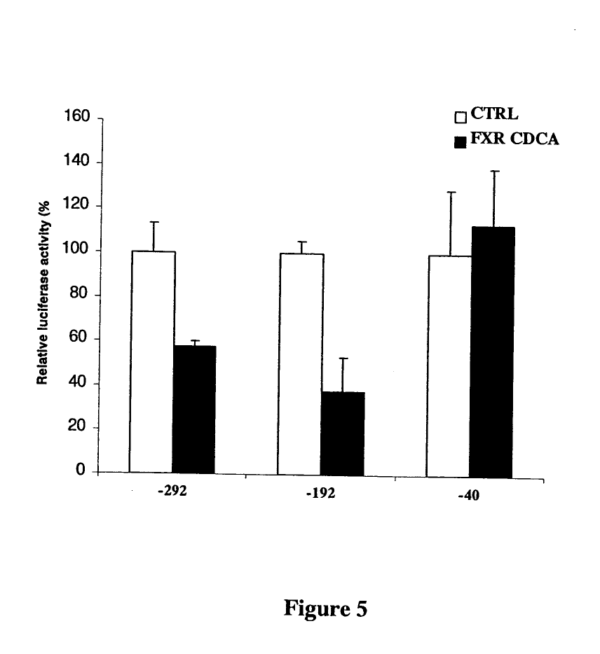

Effect of Overexpression of FXR in the Presence of CDCA on Human Apo A-I Promoter Activity in HepG2 Cells (FIG. 5)

Example 4 shows that overexpression of hFXR and mRXRα in the presence of CDCA represses the activity of fragment −254/+91 (which contains sites AB and C), [(see SEQ ID NO: 5)], fragment −192/+91 (which contains sites B and C) but not fragment −40/+91 (which contains only the minimal promoter) of the apo A-I gene promoter, cloned upstream of the luciferase reporter gene.

HepG2 cells were cotransfected by lipofection with 300 ng of pCDNA3-hFXR vector for FXR expression, 300 ng of pSG5-mRXRα vector and 1 μg of the indicated reporter vectors enabling expression of the luciferase reporter gene under the control of fragments −254/+91, −192/+91 and −40/+91 of the apo A-I promoter. These constructs were obtained by exchanging the CAT reporter gene from the previously described constructs (Ngoc Vu-Dac et al., J. Biol. Chem., 1994, 269 (49): 31012-8) with the luciferase reporter gene extracted from plasmid pGL3 from Promega (Madison, Wis., USA) as previously described (Raspé et al., J. Lipid Res., 1999, 40 (11): 2099-2110). Total DNA was adjusted to 2 μg using plasmid pBKS+. After a 3 hour transfection, cells were incubated in the culture medium with bile acids at the indicated concentrations for 36 hours. Luciferase activity was then measured as previously described (Raspé et al., J. Lipid Res., 1999, 40 (11): 2099-2110).

### Example 5

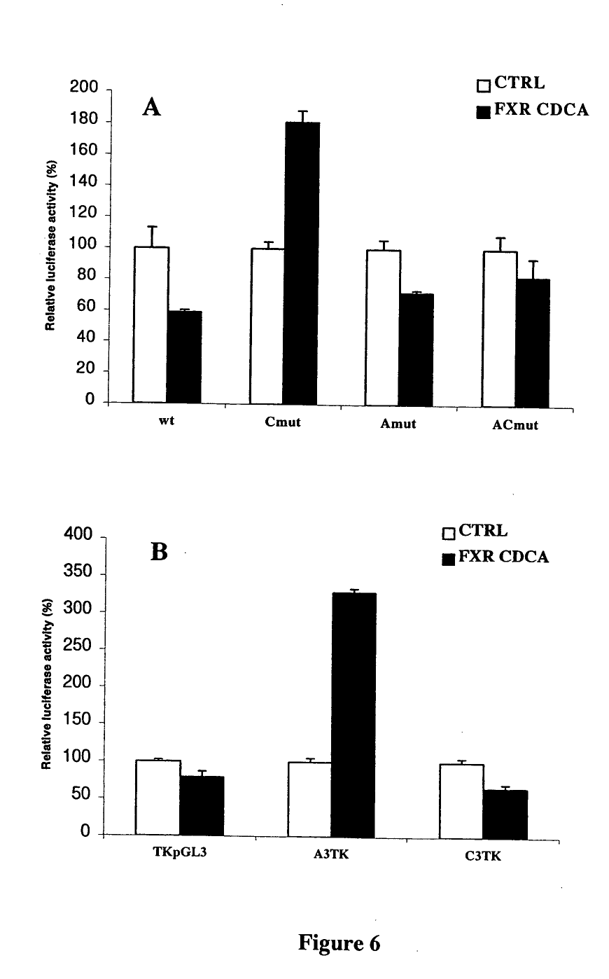

Functional Identification of FXR Response Elements Present in the Human Apo A-I Gene Promoter (FIG. 6)

Example 5 shows that overexpression of hFXR in the presence of CDCA represses the activity of wild type fragment −254/+91 of the apo A-I promoter (“wt” construct), cloned upstream of the luciferase reporter gene (panel A). It further shows that the mutation in site C present in fragment −254/+91 of the apo A-I promoter (“C mut” construct), cloned upstream of the luciferase reporter gene, results in transcriptional activation of the construct in response to overexpression of hFXR (panel A). Overexpression of hFXR in the presence of CDCA also represses the activity of fragment −254/+91 of the apo A-I promoter, cloned upstream of the luciferase reporter gene, in which site A is mutated (“A mut” construct) (panel A). Finally, a double mutation in sites A and C (“AC mut” construct) of fragment −254/+91 of the apo A-I promoter, cloned upstream of the luciferase reporter gene, results in loss of response to hFXR.

Example 5 shows that overexpression of hFXR in the presence of CDCA represses the activity of a construct containing three copies of site C of the human apo A-I promoter cloned upstream of the herpes simplex virus thymidine kinase promoter and the luciferase reporter gene (denoted “C3TK”) whereas a construct containing three copies of site A of the human apo A-I promoter cloned upstream of the herpes simplex virus thymidine kinase promoter (denoted “A3TK”) is activated by hFXR overexpression in the presence of CDCA and that the activity of a construct containing only the herpes simplex virus thymidine kinase promoter (denoted “TkpGL3”) is not affected by hFXR overexpression in the presence of CDCA (panel B).

HepG2 cells were transfected by lipofection with 300 ng of pCDNA3-hFXR vector for expression of hFXR and 1 μg of the indicated reporter vectors allowing expression of a luciferase reporter gene under the control wild type or mutated fragment −254/+91 of the human apo A-I gene promoter, under the control of the herpes simplex virus thymidine kinase promoter or under the control of three copies of site A or site C of the human apo A-I gene cloned upstream of the herpes simplex virus thymidine kinase promoter. The construct containing the wild type fragment −254/+91 of the apo A-I promoter was obtained by exchanging the CAT reporter gene of the corresponding construct described previously (Ngoc Vu-Dac et al., J. Biol. Chem., 1994, 269 (49): 31012-8) with the luciferase reporter gene extracted from plasmid pGL3 from Promega (Madison, Wis., USA), as previously described (Raspé et al., J. Lipid Res., 1999, 40 (11): 2099-2110). The construct containing fragment −254/+91 of the apo A-I promoter in which site A is mutated was obtained by directed mutagenesis of site A, in the wild type construct, using the Quick Change Site directed mutagenesis kit (Stratagene, La Jolla, Calif., USA), oligonucleotide 5′-CCCCACTGAACCCTTGATTCCTGCTTTGCAGCC-3′ (SEQ ID NO: 4) and the complementary oligonucleotide. The construct containing fragment −254/+91 of the apo A-I promoter in which site C is mutated was obtained by directed mutagenesis of site C, in the wild type construct, using the Quick Change Site directed mutagenesis kit (Stratagene, La Jolla, Calif., USA), oligonucleotide 5′-CAGAGCTATATATTATATATATAAGTTCC-3′ (SEQ ID NO: 3) and the complementary oligonucleotide. The construct containing fragment −254/+91 of the apo A-I promoter in which sites A and C are mutated was obtained by directed mutagenesis of site A, in the Cmut construct, using the Quick Change Site directed mutagenesis kit (Stratagene, La Jolla, Calif., USA), oligonucleotide 5′-CCCCACTGAACCCTTGATTCCTGCTTTGCAGCC-3′ (SEQ ID NO: 4) and the complementary oligonucleotide. The construct denoted TkpGL3 containing the herpes simplex virus thymidine kinase promoter cloned upstream of a luciferase reporter gene has been previously described (Raspé et al., J. Biol. Chem., 2001, 276 (4): 2865-2871). The C3TK construct containing three copies of site C of the human apo A-I promoter cloned upstream of the herpes simplex virus thymidine kinase promoter and the luciferase reporter gene was obtained by oriented cloning of three copies of the double-stranded oligonucleotide corresponding to the wild type site C sequence (SEQ ID NO: 1) into the SmaI site of vector TkpGL3. The A3TK construct containing three copies of site A of the human apo A-I promoter cloned upstream of the herpes simplex virus thymidine kinase promoter and the luciferase reporter gene was obtained by oriented cloning of three copies of the double-stranded oligonucleotide corresponding to the wild type site A sequence (SEQ ID NO: 2) into the SmaI site of vector TkpGL3. Total DNA was adjusted to 2 μg using plasmid pBKS+. After a 3 hour transfection, cells were incubated in the culture medium with the bile acids at the indicated concentrations for 36 hours. Luciferase activity was then measured as previously described (Raspé et al., J. Lipid Res., 1999, 40 (11): 2099-2110).

### Example 6

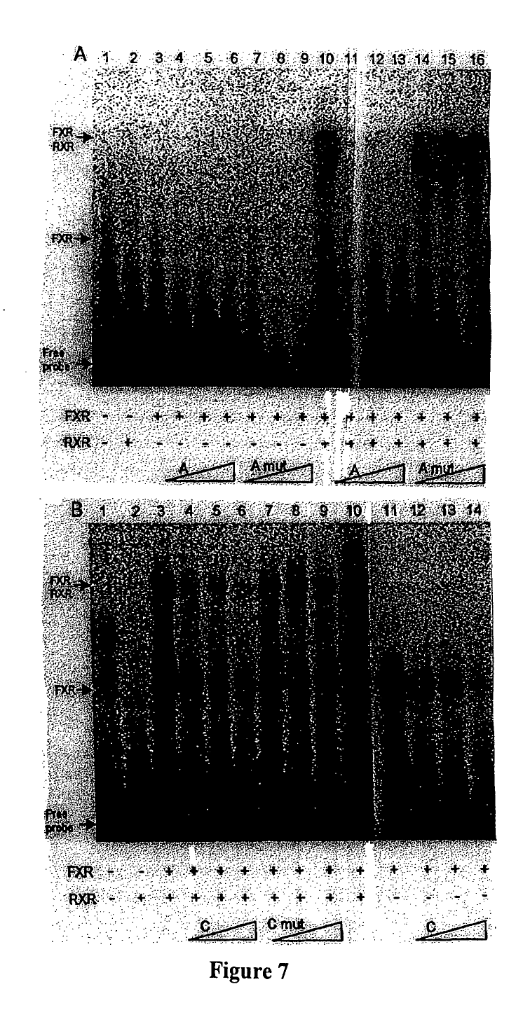

Binding of the FXR/RXR Complex to Site A (SEQ ID NO: 2) and of FXR and the FXR/RXR Complex to Site C (SEQ ID NO: 1) of the Apo A-I Promoter (FIG. 7)

Example 6 shows that the RXRα/hFXR complex binds specifically to sites A and C of the human apo A-I promoter. It also shows that FXR is capable of binding alone as a monomer only to site C of the human apo A-I promoter.

hFXR and mRXRα were produced in vitro with the TNT-T7 kit from Promega (Madison, Wis., USA) and vectors pCDNA3-hFXR and pSG5-mRXRα. Double-stranded oligonucleotides corresponding to sites A and C of the human apo A-I gene promoter (SEQ ID NO: 2, SEQ ID NO: 1) and prepared as previously described (Ngoc Vu-Dac et al., J. Biol. Chem., 1994, 269 (49): 31012-8), were labelled with [γ-³²P]-ATP using polynucleotide kinase. Two microliters of hFXR- and mRXRα-programmed reticulocyte lysate were incubated for 15 minutes at room temperature in a final volume of 20 μl of buffer containing 10 mM HEPES, 2.5 mM MgCl₂, 10% glycerol, 2.5 mg/ml BSA, 50 mM NaCl and 0.5 mM DTT with 2.5 μg of polydI-dC and 1 μg of herring sperm DNA before adding the labelled probe (0.5 ng). The mixture was incubated for 15 minutes at room temperature and the complexes were separated by electrophoresis in a non-denaturing gel using 0.25×TBE buffer. Super-shift experiments were carried out by adding anti-FXR antibody (Santa Cruz, Calif., USA) 3 hours before addition of DNA at 4° C.

### Example 7

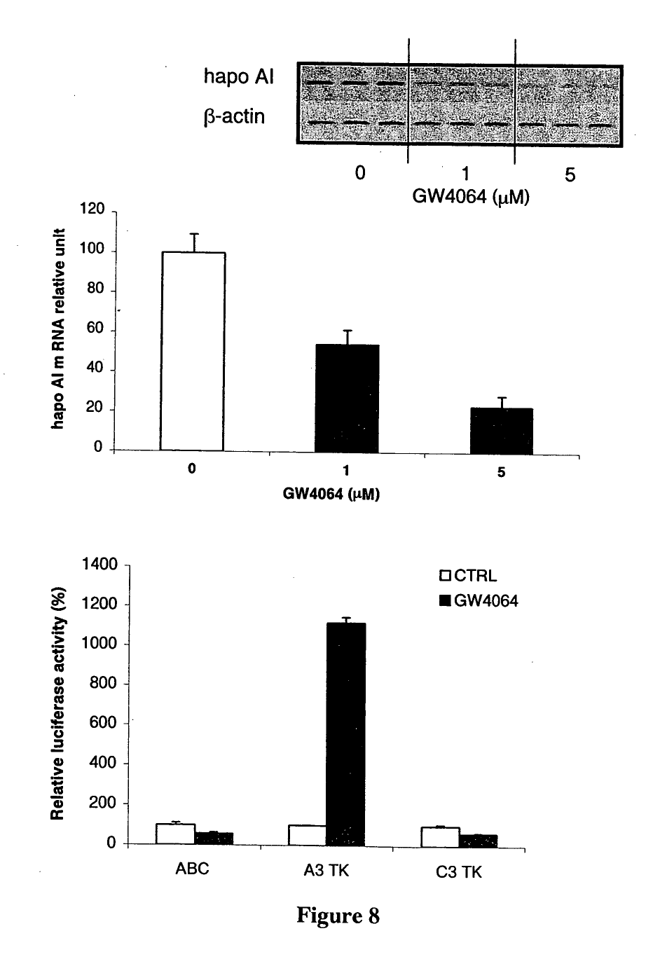

GW4064. a Synthetic Agonist of FXR, Reduces Apo A-I Expression in HepG2 Cells (FIG. 8)

HepG2 were cotransfected as described in example 5. Cellular mRNA was obtained as described above in example 2.

| | Relative apo AI mRNA (%) |
|---|---|
| Control | 100.0 ± 9.4 |
| GW4064 1 μM | 54.3 ± 7.3 |
| GW4064 5 μM | 23.0 ± 5.5 |

| | | Relative luciferase activity (%) |
|---|---|---|
| Construct ABC | Control | 100.0 ± 11.9 |
| | GW4064 1 μM | 56.8 ± 6.9 |
| Construct A3 TK | Control | 100.0 ± 1.1 |
| | GW4064 1 μM | 1119.1 ± 28.1 |
| Construct C3 TK | Control | 100.0 ± 12.6 |
| | GW4064 1 μM | 61.8 ± 2.8 |

Example 7 shows that GW4064 (Goodwin et al., Mol Cell., 2000, Sep., 6(3): 517-26), a strong specific activator of FXR, markedly reduces apo A-I expression in HepG2 cells.

Example 7 shows that overexpression of hFXR in the presence of 1 μM GW4064 represses the activity of fragment −254/+91 of the human apo A-I promoter (construct ABC) as well as that of the construct containing three copies of site C (SEQ ID NO: 1) of the human apo A-I promoter (construct C3TK). In contrast, a construct containing three copies of site A (SEQ ID NO : 2) of the human apo A-I promoter (construct A3TK) is activated by hFXR overexpression in the presence of GW4064.

1. Method for selecting, identifying or characterizing compounds capable of modulating reverse cholesterol transport, which comprises:
contacting a test compound with a nucleic acid construct containing at least one copy of an FXR response element of the human apolipoprotein A-I gene promoter or a functional variant thereof, and
determining the possible binding of said test compound to the response element.

2. Method according to claim 1, wherein the placing in contact is carried out in the presence of the FXR receptor or a functional equivalent of the FXR receptor, and wherein one determines the effect of the presence of the test compound on the binding of FXR to the response element.

3. Method for selecting, identifying or characterizing compounds capable of modulating reverse cholesterol transport, which comprises:
contacting a test compound with a host cell containing an expression cassette of a reporter gene, said cassette containing a reporter gene placed under the control of a promoter containing at least one copy of an FXR response element of the human apolipoprotein A-I gene promoter or a functional variant thereof, and
measuring the expression of the reporter gene.

4. Method according to claim 3, wherein the host cell contains the FXR receptor or a functional equivalent.

5. Method according to any one of claims 3 or 4, wherein the host cell contains a ligand of FXR.

6. Method according to any one of claims 3 to 5 comprising determining the level of reporter gene expression in the presence of the test compound or in the absence of said compound, an increase or a decrease in the level of reporter gene expression indicating the capacity of the test compound to modulate reverse cholesterol transport.

7. Method according to any one of claims 3 to 6, wherein the host cell is a mammalian cell.

8. Method according to claim 7, wherein the mammalian cell is a human cell.

9. Method according to any one of claims 1 to 8, wherein the FXR response element comprises the following sequence (SEQ ID NO: 1): 5′-CAGAGCTGATCCTTGAACTCTTAAGTTCC-3′, or a functional variant thereof capable of binding the FXR receptor.

10. Method according to any one of claims 1 to 9, wherein the FXR response element comprises the following sequence (SEQ ID NO: 2): 5′-CCCCACTGAACCCTTGACCCCTGCCCTGCAGCC-3′, or a functional variant thereof capable of binding the FXR receptor.

11. Method according to any one of claims 3 to 10, wherein the reporter gene is a gene coding for a product whose activity or presence in biological extracts can be measured, particularly one of the genes coding for luciferase, secreted alkaline phosphatase, galactosidase or lactamase.

12. Method according to any one of claims 3 to 11, wherein the promoter is chosen from among the HSV-TK promoter, the CMV immediate promoter, the PGK promoter and the promoter of the gene encoding human apolipoprotein A-I, the SV40 promoter.

13. Method according to any one of claims 1 to 12, wherein one or several compounds are tested, as a mixture or separately.

14. Method according to any one of claims 1 to 13, wherein the test compound is a combinatorial library.

15. Method according to claim 14 wherein the test compound is a clone or a library of nucleic acid clones expressing one or several DNA-binding polypeptide(s).

16. Method according to any one of the previous claims, wherein the placing in contact is carried out in a multiwell plate.

17. Method according to any one of the previous claims comprising, additionally, comparing the possible effects determined by said method with the possible effects determined by a method carried out in the same conditions but with a nucleic acid construct containing at least one mutated copy of an FXR response element of the human apolipoprotein A-I gene promoter, said mutated copy being essentially incapable of binding the FXR receptor.

18. Method according to any one of the previous claims, for selecting, identifying or characterizing compounds capable of increasing reverse cholesterol transport.

19. Method according to any one of claims 1 to 17, for selecting, identifying or characterizing compounds capable of modulating HDL activity.

20. Method according to any one of claims 1 to 17, for selecting, identifying or characterizing compounds capable of modulating the expression of apolipoprotein A-I.

21. Use of a compound capable of modulating FXR binding to the response element of the human apolipoprotein A-I gene promoter or a functional variant thereof, for preparing a composition for modulating reverse cholesterol transport.

22. Use of a compound that decreases FXR binding to the sequence SEQ ID NO: 1 or a functional variant thereof, for preparing a composition for increasing reverse cholesterol transport.

23. Use of a compound that decreases the effect of FXR on transcription of the human apolipoprotein A-I gene for preparing a composition for increasing reverse cholesterol transport.

24. Use of a compound that modulates binding of FXR and/or its cofactors to the sequence SEQ ID NO: 1 or SEQ ID NO: 2 or a functional variant thereof, for preparing a composition for modulating HDL activity.

25. Use of a compound that reduces FXR binding to the sequence SEQ ID NO: 1 or a functional variant thereof, for preparing a composition for increasing HDL activity.

26. Use of a compound that increases FXR binding to the sequence SEQ ID NO: 2 or a functional variant thereof, for preparing a composition for increasing HDL activity.

27. Use according to any one of claims 21 to 26 wherein said compound is a biological compound or a chemical compound.

28. Use according to claim 27, wherein the compound is a nuclear factor or a cofactor.

29. Use according to any one of claims 21 to 26, wherein the compound is a clone expressing one or more DNA-binding polypeptide(s).

30. Use according to any one of claims 21 to 26, wherein the compound is a compound selected, identified or characterized according to any one of claims 1 to 20.

31. Nucleic acid fragment characterized by the following sequence (SEQ ID NO: 1):
5′-CAGAGCTGATCCTTGAACTCTTAAGTTCC-3′ or a variant thereof and wherein it is capable of binding the FXR receptor.

32. Nucleic acid fragment characterized by the following sequence (SEQ ID NO: 2):
5′-CCCCACTGAACCCTTGACCCCTGCCCTGCAGCC-3′ or a variant thereof and wherein it is capable of binding the FXR receptor.

33. Nucleic acid construct containing at least one copy of the nucleic acid fragment according to claim 31 and/or the nucleic acid fragment according to claim 32.

34. Nucleic acid construct containing at least one mutated copy of the nucleic acid fragment according to claim 31 and/or the nucleic acid fragment according to claim 32, said mutated copy being essentially incapable of binding the FXR receptor.

35. Expression cassette containing at least one copy of the nucleic acid fragment according to claim 31 and/or 32, said copy or copies being contained in a promoter associated with a reporter gene placed under the control of said promoter.

36. Expression cassette containing at least one mutated copy of the nucleic acid fragment according to claim 31 and/or 32, said copy being contained in a promoter associated with a reporter gene placed under the control of said promoter.

37. Cassette according to any one of claims 35 or 36, wherein said promoter is chosen from among the HSV-TK promoter, the CMV immediate promoter, the PGK promoter, the SV40 promoter and the human apolipoprotein A-I gene promoter.

38. Cassette according to any one of claims 35 to 37, wherein the reporter gene is a gene whose activity or presence in biological extracts can be measured.

39. Cassette according to claim 38, wherein the reporter gene is the gene coding for luciferase, secreted alkaline phosphatase, galactosidase or lactamase.

40. Host cell containing a construct or cassette according to any one of claims 33 to 39.

41. Utilization of a construct or cassette according to any one of claims 33 to 39 or of a cell according to claim 40, for in vitro screening of compounds capable of modulating HDL activity.

42. Pharmaceutical composition comprising a compound selected, identified or characterized according to one of the methods in any one of claims 1 to 20.
